(19) United States
(12) Patent Application Publication
(10) Pub. No.: US 2014/0178989 A1
(43) Pub. Date: Jun. 26, 2014

(54) INDUCED PLURIPOTENT STEM CELLS FROM HUMAN UMBILICAL CORD TISSUE-DERIVED CELLS

(71) Applicant: DePuy Synthes Products, LLC, Raynham, MA (US)

(72) Inventors: Charito Buensuceso, North Brunswick, NJ (US); Agineszka Seyda, Belle Mead, NJ (US); David C. Colter, Hamilton, NJ (US); Sridevi Dhanaraj, Raritan, NJ (US); Brian C. Kramer, Plainfield, NJ (US)

(21) Appl. No.: 14/085,845

(22) Filed: Nov. 21, 2013

Related U.S. Application Data
(63) Continuation of application No. 13/330,931, filed on Dec. 20, 2011.

(51) Int. Cl. C12N 5/073

(52) U.S. Cl. CPC C12N 5/0605
USPC 435/366

(57) ABSTRACT

We have disclosed an induced pluripotent stem cell and the method of preparing the induced pluripotent stem cell from a human umbilical cord tissue-derived cell. More particularly, we have disclosed a human umbilical cord tissue-derived iPS cell which may be differentiated into cells of ectoderm, mesoderm, and endoderm lineages.

### FIELD OF THE INVENTION

The invention relates to induced pluripotent stem cells. More particularly, the invention relates the reprogramming of human umbilical cord tissue-derived cells (hUTC) into induced pluripotent stem (iPS) cells.

### BACKGROUND OF THE INVENTION

Induced pluripotent stem (iPS) cells have generated interest for application in regenerative medicine, as they allow the generation of patient-specific progenitors in vitro having a potential value for cell therapy (Takahashi, K. and Yamanaka, S., Cell 126, 663-76 (2006)). However, in many instances an off-the-shelf approach would be desirable, such as for cell therapy of acute conditions or when the patient's somatic cells are altered as a consequence of a chronic disease or ageing.

Ectopic expression of pluripotency factors and oncogenes using integrative viral methods is sufficient to induce pluripotency in both mouse and human fibroblasts (Takahashi, K. and Yamanaka, S., Cell 126, 663-76 (2006); Takahashi, K. et al. Cell 131,861-72 (2007); Hochedlinger, K. and Plath, K., Development 136,509-23 (2009); Lowry, W. E. et al., Proc Natl Acad Sci USA 105, 2883-8 (2008)). However, this process is slow, inefficient and the permanent integration of the vectors into the genome limits the use of iPS cells for therapeutic applications (Takahashi, K. and Yamanaka, S., Cell 126, 663-76 (2006)). Further studies have shown that the age, origin, and cell type used has a deep impact on the reprogramming efficiency. Recently, it was shown that retroviral transduction of human keratinocytes resulted in reprogramming to pluripotency which was 100-fold more efficient and twice as fast when compared to fibroblasts. It was hypothesized that these differences could result from the endogenous expression of KLF4 and c-MYC in the starting keratinocyte population and/or the presence of a pool of undifferentiated progenitor cells presenting an epigenetic status more amenable to reprogramming (Lowry, W. E. et al., Proc Natl Acad Sci USA 105, 2883-8 (2008).). This latter hypothesis has been further supported by other studies in mouse. (Silva, J. et al., PLoS Bio16, e253 (2008); and Eminli, S. et al., Stem Cells 26, 2467-74 (2008)). However, stem cells are usually rare and difficult to access and isolate in large amounts (e.g., neural stem cells) (Kim, J. B. et al., Cell 136, 411-9 (2009); Kim, J. B. et al., Nature 454, 646-50 (2008)).

Human umbilical cord tissue-derived iPS cells represent a viable supply of pluripotent cells for a number of applications. It is of particular interest to regenerative medicine because umbilical cord tissue is from an early developmental origin and is has been shown to possess multilineage differentiation potential. In addition, umbilical cord tissue is likely exempt from incorporated mutations when compared with juvenile or adult donor cells such as skin fibroblasts or keratinocytes.

### SUMMARY OF THE INVENTION

We describe herein, an induced pluripotent stem cell prepared by reprogramming a human umbilical cord tissue-derived cell. The human umbilical cord tissue-derived cell is an isolated umbilical cord tissue cell isolated from human umbilical cord tissue substantially free of blood that is capable of self-renewal and expansion in culture, has the potential to differentiate into cells of other phenotypes, can undergo at least 40 doublings in culture, maintains a normal karyotype upon passaging, and has the following characteristics: expresses each of CD10, CD13, CD44, CD73, CD90, PDGFr-alpha, PD-L2, and HLA-A,B,C; does not express any of CD31, CD34, CD45, CD80, CD86, CD117, CD141, CD178, B7-H2, HLA-G, or HLA-DR,DP,DQ; and increased expression of a gene for each of interleukin 8; reticulon 1; and chemokine (C-X-C motif) ligand 3 relative to that of a human cell which is a fibroblast, a mesenchymal stem cell, or an iliac crest bone marrow cell. The human umbilical cord tissue-derived cell further has the following characteristics: secretes each of the factors MCP-1, MIP1beta, IL-6, IL-8, GCP-2, HGF, KGF, FGF, HB-EGF, BDNF, TPO, RANTES and TIMP1; and does not secrete any of the factors SDF-1alpha, TGF-beta2, ANG2, PDGFbb, MIP1a and VEGF.

### BRIEF DESCRIPTION OF THE FIGURES

FIG. 1. Morphology of human umbilical cord tissue-derived iPS cells, clone K1, obtained from transduction of hUTC with human OCT4, SOX2, KLF4, and c-MYC and shRNA to p53. Clones are shown on irradiated mouse embryonic fibroblast (MEF) feeder layer at passage 1.

FIG. 2. Human umbilical cord tissue-derived iPS cells (clone K1) grown on MEF feeder layer and stained for alkaline phosphatase (4× magnification).

### DETAILED DESCRIPTION OF THE INVENTION

We disclose herein, the reprogramming of human umbilical cord tissue-derived cells (hUTC) to pluripotency by retroviral transduction of four (OSKM) transcription factors with or without the downregulation of p53. Using the methods and compositions described herein, hUTC are reprogrammed to pluripotency by retroviral transduction with OCT4, SOX2, KLF4, and c-MYC. The resulting reprogrammed hUTC have the characteristics of induced pluripotent stem (iPS) cells.

In one embodiment, an induced pluripotent stem (iPS) cell is prepared from a human umbilical cord tissue-derived cell, referred to herein as a human umbilical cord tissue-derived iPS cell. The hUTC were reprogrammed by the forced expression of the reprogramming factors in the presence or absence of shRNA to p53. The reprogrammed cells were characterized for morphology, staining for alkaline phosphatase, expression of pluripotency markers, methylation of specific promoters, and expression of specific germ layer markers.

hUTC are a unique population of cells isolated from human umbilical cord tissue. The methods for isolating hUTC are described in U.S. Pat. No. 7,510,873, incorporated by reference herein in its entirety. Briefly, the method comprises (a) obtaining human umbilical cord tissue; (b) removing substantially all of the blood to yield a substantially blood-free umbilical cord tissue, (c) dissociating the tissue by mechanical or enzymatic treatment, or both, (d) resuspending the tissue in a culture medium, and (e) providing growth conditions which allow for the growth of a human umbilical cord tissue-derived cell capable of self-renewal and expansion in culture and having the potential to differentiate into cells of other phenotypes.

In preferred embodiments, the cells do not express telomerase (hTert). Accordingly, one embodiment the human umbilical cord tissue-derived cells that do not express telomerase (hTert) and that have one or more of the characteristics disclosed herein.

In one embodiment, the cells are umbilical cord tissue-derived cells which are isolated from human umbilical cord tissue substantially free of blood, are capable of self-renewal and expansion into culture, have the potential to differentiate into cells of other phenotypes, can undergo at least 40 doublings, and have the following characteristics: (a) express each of CD10, CD13, CD44, CD73, CD90, PDGFr-alpha, PD-L2 and HLA-A,B,C; (b) do not express any of CD31, CD34, CD45, CD80, CD86, CD 117, CD141, CD178, B7-H2, HLA-G, or HLA-DR,DP,DQ; and (c) increased expression of interleukin-8; reticulon 1; and chemokine receptor ligand (C-X-C motif) ligand 3, relative to that of a human cell which is a fibroblast, a mesenchymal stem cell, or an iliac crest bone marrow cell. In one embodiment, these umbilical cord derived cells also have one of more of the following characteristics: (a) secretion of each of the factor MCP-1, MIP1beta, IL-6, IL-8, GCP-2, HGF, KGF, FGF, HB-EGF, BDNF, TPO, RANTES, and TIMP1; and (b) no secretion of any of the factors SDF-1alpha TGF-beta2, ANG2, PDGFbb, MIP1a and VEGF. In another embodiment, these umbilical cord tissue-derived cells do not express hTERT or telomerase.

In another embodiment, the cells are umbilical cord tissue-derived cells which are isolated from human umbilical cord tissue substantially free of blood, are capable of self-renewal and expansion into culture, have the potential to differentiate into cells of other phenotypes, do not express CD117 and express telomerase or hTert. In yet another embodiment, the cells further do not express CD45. In an alternate embodiment, the cells further do not express any of CD31, CD34, CD80, CD86, CD141, CD178, B7-H2, HLA-G, or HLA-DR,DP,DQ. In another alternate embodiment, the cells further express each of CD10, CD13, CD44, CD73, CD90, PDGFr-alpha, PD-L2 and HLA-A,B,C. In yet another embodiment of the invention, the cells further can undergo at least 40 doublings. In yet another embodiment, the cells further show increased expression of interleukin-8; reticulon 1; and chemokine receptor ligand (C-X-C motif) ligand 3, relative to that of a human cell which is a fibroblast, a mesenchymal stem cell, or an iliac crest bone marrow cell. In yet another embodiment, the cells further have each of the following characteristics: (a) secretion of each of the factor MCP-1, MIP1beta, IL-6, IL-8, GCP-2, HGF, KGF, FGF, HB-EGF, BDNF, TPO, RANTES, and TIMP1 and (b) no secretion of any of the factors SDF-1alpha TGF-beta2, ANG2, PDGFbb, MIP1a and VEGF.

The hUTC were reprogrammed using viral reprogramming methods. In one embodiment, the hUTC were transfected with retroviruses individually carrying constitutively expressed human transcription factors OCT4, SOX2, KLF4, and c-MYC. Briefly, hUTC were plated on a 6-well plate, at 1×10⁵ cells per well in hFib medium, and incubated for 6 hours at 5% CO₂ and 37° C. The four murine retroviral constructs (OCT4, SOX2, KLF4, and c-MYC) and an agent for increasing the efficiency of transfection were added to each well. After overnight incubation at 5% CO₂ and 37° C., this transduction step was repeated. After 24 hours, the medium was aspirated and fresh hFib medium was added. After another 48 hours, cells were harvested and plated on a 60-mm dish pre-seeded with mouse embryonic feeder (MEF) cells in hFib medium. After 48 hours, medium was replaced with hES medium. Cells were allowed to incubate for three to four weeks with hES medium replaced daily.

In another embodiment, hUTC were transfected with VSVg murine retroviruses individually carrying constitutively expressed human transcription factors OCT4, SOX2, KLF4, and c-MYC and p53-shRNA. The inhibition of p53 has been previously shown to enhance the reprogramming efficiency of specific cell types presumably by slowing down cell proliferation (Zhao Y et al., (2008) Cell Stem Cell 3: 475-479; Sarig, R., et al., J. Exp. Med. 207: 2127-2140 (2010)). Briefly, hUTC were plated in a 6-well plate, at 1×10⁵cells per well in Hayflick medium and incubated overnight at 5% CO₂ and 37° C. For viral transfections, transduction medium having the four VSVg murine retroviral constructs (OCT4, SOX2, KLF4, and c-MYC) and p53-shRNA and an agent for increasing the efficiency of transfection was prepared for each well. Medium was aspirated from the wells, transduction medium was added, and incubated overnight at 5% CO₂ and 37° C. This transduction step was repeated the following day and after overnight incubation, the transduction medium was replaced with Hayflick medium. Cells were allowed to incubate for another four days with Hayflick medium replaced every two days.

The transfected hUTC were then cultured and observed for the appearance of classical iPS cell morphology. Classical iPS cell morphology refers to the formation of tightly packed cell colonies that are refractive or “shiny” under light microscopy with very sharp and well-defined edges. Cells exhibiting classical iPS cell morphology were isolated, subcultured, and expanded to provide human umbilical cord tissue-derived iPS cells.

Several criteria are used to assess whether iPS cells are fully reprogrammed including morphology (as described above), staining for alkaline phosphatase, expression of pluripotency markers, methylation of specific promoters, and expression of specific germ layer markers. The expression of a key pluripotency factor, NANOG, and embryonic stem cell specific surface antigens (SSEA-3, SSEA-4, TRA1-60, TRA1-81) have been routinely used to identify fully reprogrammed human cells. At the functional level, iPS cells also demonstrate the ability to differentiate into lineages from all three embryonic germ layers.

The human umbilical cord tissue-derived iPS cell prepared by the methods described herein was characterized for pluripotency. These cells which display the classical iPS cell morphology, are capable of self-renewal, express the key pluripotency markers (TRA1-60, TRA1-81, SSEA3, SSEA4, and NANOG), demonstrate differentiation into lineage from three germ layers, and show normal karyotype.

Human umbilical cord tissue-derived iPS cells represent a good source of pluripotent cells for regenerative medicine. With this technology, it is now possible to generate pluripotent cells in large numbers. Another important benefit is the potential to obtain iPS cells from a tissue originating from an early developmental origin and from a tissue that is probably free from incorporated mutations relative to adult donor cells. These cells will be useful for comparisons among iPS cells derived from multiple tissues regarding the extent of the epigenetic reprogramming, differentiation ability, stability of the resulting lineages, and the risk of associated abnormalities.

The invention is further explained in the description that follows with reference to the drawings illustrating, by way of non-limiting examples, various embodiments of the invention.

### EXAMPLES

### Example 1

### Reprogramming of hUTC into iPS Cells

hUTC obtained according to the methods described in U.S. Pat. No. 7,510,873, were transduced with murine retroviruses individually carrying constitutively expressed human transcription factors (OCT4, SOX2, KLF4, and c-MYC). hUTC were thawed and cultured for one passage before transduction. On day 1, hUTC were trypsinized and plated onto 6-well plates at 1×10⁵ cells per well in 2 milliliters of hFib medium (DMEM (Invitrogen Corporation, Carlsbad, Calif., catalog number 11965-092) containing 10% fetal bovine serum (FBS) sold under the tradename BENCHMARK (Gemini Bio-products, West Sacramento, Calif., catalog number 100-106, vol/vol), 2 millimolar L-glutamine sold under the tradename GLUTAMAX (Invitrogen Corporation, catalog number 35050-061), 50 Units/millilter penicillin and 50 milligrams/milliliter streptomycin (Invitrogen Corporation, catalog number 15140-122) per well. Cells were incubated for 6 hours at 5% CO₂ and 37° C. Medium was aspirated to remove non-viable cells and 2 milliliters of fresh hFib medium was added. Retroviruses individually carrying OCT4, SOX2, KLF4 and c-MYC (each with an MOI of 5) and 10 microliters (200×) of an infection reagent sold under the tradename TRANSDUX (System Biosciences, Inc., Mountain View, Calif., catalog number LV850A-1) were added into each well, and mixed gently by swirling the plate. On day 2, the viral transduction step was repeated. On day 3, the transduction medium was removed, the cells washed, and the medium was replaced with 2 milliliters of hFib medium. On this same day, 1×10⁵ mitomycin C-treated MEF cells were seeded onto 60-millimeter dishes (pre-coated with 0.1% gelatin (Millipore Corporation, Billerica, Mass., catalog number ES-006-B, wt/vol) and incubated overnight at 5% CO₂ and 37° C.

To monitor the formation of reprogrammed or iPS cell colonies, the transduced hUTC were harvested by trypsinization on day 4, resuspended in hES medium (DMEM/F12, Invitrogen Corporation, catalog number 11330-32) containing 20% knock-out serum (KSR, Invitrogen Corporation, catalog number 10828-028, vol/vol), 10 nanograms/millilter basic fibroblast growth factor (bFGF; R&D Systems, Inc., Minneapolis, Minn., catalog number 233-FB-025), 1 millimolar GLUTAMAX , 0.1 millimolar nonessential amino acids (Invitrogen Corporation, catalog number 11140-050), 0.1 millimolarM 2-mercaptoethanol (Sigma-Aldrich, St. Louis, Mo., catalog number M7522), 50 Units/milliliter penicillin and 50 milligrams/milliliter streptomycin(Invitrogen Corporation, catalog number 15140-122) and then plated on mouse embryonic fibroblast (MEF) feeder plate at a concentration of 1×10⁶ cells per 60 millimeter dish. Cells were plated at different cell densities between 3×10⁴ to 1×10⁵ cells. On day 6, medium was aspirated and replaced with hES medium. Medium was changed with fresh hES medium daily for 3 to 4 weeks. The plates were checked daily to identify iPS cell colonies.

For reprogramming in the presence of shRNA to p53, hUTC were transduced with retroviral constructs specifically, VSVg murine retroviruses individually carrying constitutively expressed human transcription factors (OCT4, SOX2, KLF4, and c-MYC) and VSVg murine retrovirus containing p53-shRNA.

The murine retroviruses were produced using the 293-gp2 retrovirus packaging cells that were plated one day prior to transfection onto 6 centimeter dishes at a density of 3×10⁶ cells per dish and incubated overnight at 5% CO₂ and 37° C. Each dish was then transfected with 3 micrograms pMX vector (Sox2, Oct4, cMyc, Klf4, or p53-shRNA vector, 1 microgram VSV-g and 16 microliters of a transfection agent sold under the tradename FUGENE HD (Roche Applied Bioscience, Indianapolis, Ind., catalog number 04709705001) according to the manufacturer's standard protocol. Viruses were then collected 48 hours after transfection and filtered through a 0.45micron filter prior to use.

hUTC were thawed and cultured for one passage before transduction. One day before transduction, hUTC were trypsinized and plated onto 2 wells of a 6-well plate at 1×10⁵ cells per well in 2 milliliters of renal epithelial growth medium (REGM, Lonza Walkersville, Inc., Walkersville, Md.) per well. Cells were incubated overnight at 5% CO₂ and 37° C. On day 1, 2.5 milliliters of transduction medium was prepared for each well containing 500 microliters of each freshly-made virus and 4 nanograms/milliliter of polybrene. The culture medium was aspirated from the wells, the transduction medium was added, and was incubated overnight at 5% CO₂ and 37° C. On day 2, the viral transduction step was repeated. On day 3, the transduction medium was removed and replaced with REGM. Media changes were performed every 2 days until day 7.

To monitor the formation of reprogrammed or iPS cell colonies, the transduced hUTC were harvested by trypsinization, resuspended in culture medium sold under the tradename STEMEDIUM NUTRISTEM (Stemgent, Inc., Cambridge, Mass., catalog number 01-0005) supplemented with an additional 20 nanograms/milliliter of bFGF (iPS-Nu medium) or standard knockout serum replacement (KSR)-containing human ES medium with 20 nanograms/milliliter of bFGF (iPS-KSR medium), and then plated on a basement membrane matrix, sold under the tradename MATRIGEL (BD Biosciences, Chicago, Ill., catalog number 354277)-coated or mouse embryonic fibroblast (MEF) feeder plate at a concentration of 1×10⁴ cells per well in 6-well plate. Medium was changed with fresh iPS medium every 2 days during the first week and daily during weeks 2 to 6. The plates were checked daily to identify iPS cell colonies.

Colonies exhibiting the ‘classic’ reprogrammed or iPS cell morphology were manually picked from MEF feeder plates and seeded onto a single well of a 12-well MEF feeder plate. Culture medium was changed daily. After 4-6 days, the colonies were manually picked from the 12-well plates and expanded into 6-well plates. Culture medium was changed daily and manually split 1:3 every 4-6 days. Cells from each well were frozen at various stages in using a freezing medium, sold under the tradename CRYOSTEM (Stemgent, Inc., catalog number 01-0013).

### Results

Reprogramming of hUTC with the retroviruses expressing the four reprogramming factors resulted in reprogrammed colonies exhibing the iPS cell morphology. Reprogrammed colonies were manually picked and of these colonies, 12 were expanded and frozen. Human umbilical cord tissue-derived iPS cells obtained using the four reprogramming factors are denoted as FF followed by the colony number.

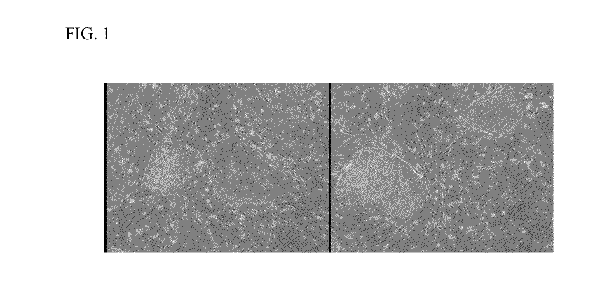

Reprogramming of hUTC with the retroviruses expressing the four reprogramming factors and shRNA to p53 resulted in reprogrammed colonies exhibing the iPS cell morphology. Twenty-five reprogrammed colonies were manually picked and of these colonies, 19 were expanded and frozen. Human umbilical cord tissue-dervied iPS cells obtained using the four reprogramming factors and p53 shRNA are denoted as N (originally maintained in STEMEDIUM NUTRISTEM-containing medium) followed by the colony number or as K (originally maintained in KSR-containing medium) followed by the colony number (FIG. 1).

### Example 2

### Expression of Pluripotency Markers

The human umbilical cord tissue-derived iPS cells prepared in Example 1 were assessed for their expression of pluripotency markers by immunocytochemistry. Following fixation of the colonies in 4% paraformaldehyde, immunofluorescent staining for pluripotency markers was performed using the antibody reagents shown in Table 1(all antibodies were obtained from Stemgent, Inc.).

TABLE 1

| Marker | Primary Antibody | Secondary Antibody |
|---|---|---|
| TRA-1-81 | Mouse anti-Human TRA-1-81 | NA |
| | Antibody, sold under the | |
| | tradename DYLIGHT 549, | |
| | catalog number 09-0082 | |
| TRA-1-60 | Mouse anti-Human TRA-1-60 | NA |
| | Antibody, sold under the | |
| | tradename STAINALIVE | |
| | DYLIGHT 488, catalog | |
| | number 09-0068 | |
| SSEA-3 | Anti-Human SSEA-3 | Goat anti-Rat IgG + |
| | Antibody, catalog | IgM Antibody, sold under |
| | number 09-0014 | the tradename CY 3, |
| | | catalog number 09-0038 |
| SSEA-4 | Anti-Human SSEA-4 | Goat anti-Mouse IgG + |
| | Antibody, catalog | IgM Antibody, sold under |
| | number 09-0006 | the tradename CY 3, |
| | | catalog number 09-0036 |
| NANOG | Anti-Mouse/Human | Goat anti-Rabbit IgG |
| | NANOG Antibody, | Antibody, sold under the |
| | catalog number 09-0020 | tradename CY 3, catalog |
| | | number 09-0037 |

### Results

A representative human umbilical cord tissue-derived iPS cells clone, clone K1, was assessed for expression of pluripotency markers. Human umbilical cord tissue-derived iPS cells, clone K1, express the markers TRA1-60, TRA1-81, SSEA3, SSEA4, and NANOG. These markers were not detected in the parental hUTC. The expression of these markers indicates pluripotency of the human umbilical cord tissue-derived iPS cells.

### Example 3

### Methylation Analysis of Oct4, Nanog, and Sox2 Promoters

The human umbilical cord tissue-derived iPS cells prepared in Example 1, clone N1, were analyzed for the methylation status of the Oct4, Nanog, and Sox2 promoter regions using the bisulfite sequencing method and was performed by Seqwright, Inc. (Houston, Tex.). The bisulfite method is the most commonly used technique for identifying specific methylation patterns within a DNA sample. It consists of treating DNA with bisulfite, which converts unmethylated cytosines to uracil but does not change methylated cytosines. It is used both for loci-specific or genome-wide analyses.

Approximately 100 to 500 bp-long promoter regions of of Oct4, Nanog, and Sox2 were examined for methylation patterns. DNA (see Table 2) were prepared using the DNA extraction kit sold under the tradename DNEASY (Qiagen, Inc., Valencia, Calif., catalog number 69506) and were sent to Seqwright, Inc. for analysis.

TABLE 2

| Sample ID | Sample description |
|---|---|
| 1 | parental hUTC |
| 2 | hUTC N1 p12 |

### Results:

Table 3 summarizes the results obtained from the analysis of the promoter regions. Within the regions that were tested, no methylation sites were detected within the Sox2 promoter. There were 5 methylation sites detected for the Oct4 promoter and 2 methylation sites for the Nanog promoter. Relative to the parental cells, the umbilical cord tissue-derived iPS cells showed a change in the methylation pattern in 1 of the 5 sites within the Oct4 promoter and in 1 of the 2 sites for the Nanog promoter. This change in methylation pattern is a characteristic of iPS cells.

TABLE 3

| | | Total | | |
|---|---|---|---|---|
| | | methylation sites | Total | Total |
| Promoter | Bp | found in the | changed | unchanged |
| region | examined | region | sites | sites |
| Oct4 promoter | ~520 bp | 5 | 1 | 4 |
| Nanog promoter | ~100 bp | 2 | 1 | 1 |
| Sox2 promoter | ~550 bp | 0 | — | — |

### Example 4

### Alkaline Phosphatase Staining

The pluripotency of the human umbilical cord tissue-derived iPS cells prepared in Example 1, clone K1, was also assessed by alkaline phosphatase staining (AP) and was performed using an alkaline phosphatase detection kit (Millipore Corporation, Billerica, Mass., catalog number SCR004). Human umbilical cord tissue-derived iPS cells were plated onto MEF-seeded 24-well plates and maintained in a 37° C. incubator. After 3-5 days, culture media was aspirated from the wells and the cells were fixed using 4% paraformaldehyde for 1-2 minutes. The fixative was removed and the cells were washed with 1 milliliter of 1× rinse buffer. Afterwards, rinse buffer was replaced with 0.5 milliliter of staining reagent mix and incubated at room temperature for 15 minute. The staining reagent was prepared by mixing the kit components fast red violet (FRV) and naphthol AS-BI phosphate solution with water in a 2:1:1 ratio (FRV:Naphthol:water) in an aluminum foil-covered tube. The staining reagent was removed and cells were washed once with 1 milliliter of 1× rinse buffer and then incubated in 0.5 milliliter of PBS. Images of stained cells were captured with a photomicroscope. Cells exhibiting AP activity appear purple.

### Results

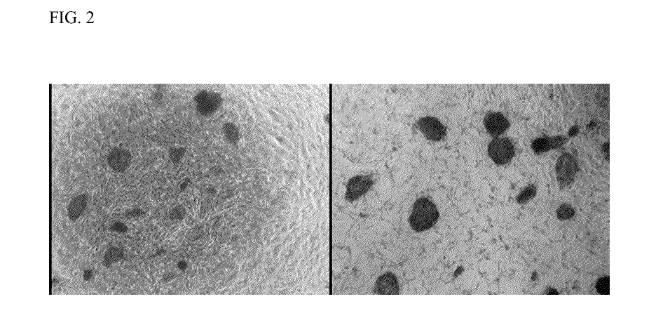

As shown in FIG. 2, human umbilical cord tissue-derived iPS cells, clone K1, exhibited positive alkaline phosphatase staining that is indicative of the pluripotent state.

### Example 5

### Differentiation into Lineages of Three Germ Layers

The differentiation capacity of the human umbilical cord tissue-derived iPS cells prepared in Example 1, clone FF44, into ectodermal, mesodermal, and endodermal lineages was evaluated by staining for markers specific to the three germ layers.

Human umbilical cord tissue-derived iPS cells were seeded onto MATRIGEL basement membrane matrix-coated plates in MEF conditioned medium for seven days. Immunocytochemistry of the differentiated human umbilical cord tissue-derived iPS cells was performed by fixing the cells in 4% paraformaldehyde for 10 minutes at room temperature. Fixed cells were washed twice with phosphate-buffered saline (PBS), and incubated at room temperature for one hour in a PBS+3% fetal bovine serum solution. Afterwards, cells were washed twice with a washing buffer sold under the tradename BD PERM/WASH (BD Biosciences, Chicago, Ill., catalog number SI-2091KZ). The cells were incubated in the specific antibody (Table 4) in BD PERM/WASH overnight at 4° C. Cells were washed five times with BD PERM/WASH and then incubated with the secondary antibody for 1.5-2 hours at room temperature in the dark. After washing the cells with PBS, cell nuclei were visualized by incubating the cells in 0.1-1 microgram/milliliter API (DNA stain, 1:10000 diluted) for 2 min. After a final wash with PBS, the cells were processed for immunofluorescence microscopy.

TABLE 4

| Germ | | |
|---|---|---|
| Layer | Primary Antibody | Secondary Antibody |
| Ectoderm | Nestin (Stemgent, Inc., | Goat anti-mouse IgG antibody, |
| | catalog number 09-0045) | sold under the tradename |
| | | ALEXA FLUOR 680, |
| | | (Invitrogen Corporation, |
| | | Carlsbad, CA, catalog |
| | | number A20983) |
| Mesoderm | Alpha-smooth muscle | Goat anti-mouse IgG antibody, |
| | actin (SMA; Sigma- | sold under the tradename |
| | Aldrich, St. Louis, MO, | ALEXA FLUOR 680, |
| | catalog number | (Invitrogen Corporation, |
| | SAB1400414) | catalog number A20983) |
| Endoderm | Alpha-fetoprotein1 (AFP1; | FITC Goat anti-rabbit IgG |
| | Dako North America, Inc., | (Abcam, Cambridge, MA, |
| | Carpinteria, CA, catalog | catalog number Ab6717) |
| | number A0008) | |

### Results

The human umbilical cord tissue-derived iPS cells were stained with antibodies to nestin, alpha-smooth muscle actin (alpha-SMA), and alpha-fetoprotein 1(AFP1) to evaluate differentiation into ectodermal, mesodermal, and endodermal lineages, respectively. The human umbilical cord tissue-derived iPS cell, clone K1, expressed these germ layer markers indicating that these cells have the capacity to differentiate into cells from these germ layers.

### Summary

Overall,we have shown the generation of human umbilical cord tissue-derived iPS cells by overexpression of human transcription factors using integrating (viral) methods. These results demonstrate that human umbilical cord tissue-derived iPS cells express the pluripotency markers TRA1-60, TRA1-81, SSEA3, S SEA4, and NANOG and exhibit positive alkaline phosphatase staining Upon examination of a 100-500 base pair region of the Oct4 promoter, the human umbilical cord tissue-derived iPS cells show a change in methylation on 1 out of the 5 methylation sites examined compared with the parental hUTC line. For the Nanog promoter, the human umbilical cord tissue-derived iPS cells show a change in methylation on 1 out of the 2 methylation sites examined compared with the parental hUTC line.

These cells also display protein markers of cells derived from ectodermal, mesodermal, and endodermal lineages showing the differentiation potential of these reprogrammed cells.

While the invention has been described and illustrated by reference to particular embodiments and examples, those of ordinary skill in the art will appreciate that the invention lends itself to variations not necessarily illustrated herein. For this reason, then, reference should be made solely to the appended claims for purposes of determining the true scope of the invention.

1-7. (canceled)

8. A human umbilical cord tissue-derived cell wherein the human umbilical cord tissue-derived cell is an isolated umbilical cord tissue cell isolated from human umbilical cord tissue substantially free of blood that is capable of self-renewal and expansion in culture, has the potential to differentiate into cells of other phenotypes, can undergo at least 40 doublings in culture, maintains a normal karyotype upon passaging, has the following characteristics: expresses each of CD10, CD13, CD44, CD73, CD90, PDGFr-alpha, PD-L2, and HLA-A,B,C; does not express any of CD31, CD34, CD45, CD80, CD86, CD117, CD141, CD178, B7-H2, HLA-G, or HLA-DR,DP,DQ; and increased expression of a gene for each of interleukin 8; reticulon 1; and chemokine (C-X-C motif) ligand 3 relative to that of a human cell which is a fibroblast, a mesenchymal stem cell, or an iliac crest bone marrow cell, and is transfected with murine retroviruses, individually carrying constitutively expressed human transcription factors OCT4, SOX2, KLF4, and c-MYC.

9. The human umbilical cord tissue-derived cell of claim 1 wherein the murine retrovirus further carries p53-shRNA.

10. The human umbilical cord tissue-derived cell of claim 1 wherein the human umbilical cord tissue-derived cell further has the following characteristics: secretes each of the factors MCP-1, MIP1beta, IL-6, IL-8, GCP-2, HGF, KGF, FGF, HB-EGF, BDNF, TPO, RANTES and TIMP1; and
does not secrete any of the factors SDF-1alpha, TGF-beta2, ANG2, PDGFbb, MIP1a and VEGF.

11. The human umbilical cord tissue-derived cell of claim 1 wherein the human umbilical cord tissue-derived cell expresses TRA1-60, TRA1-81, SSEA3, SSEA4, and NANOG.

12. The human umbilical cord tissue-derived cell of claim 1 wherein the human umbilical cord tissue-derived cell is positive for alkaline phosphatase staining.

13. The human umbilical cord tissue-derived cell of claim 1 wherein the human umbilical cord tissue-derived cell differentiates into cells of ectoderm, mesoderm, and endoderm lineages.
